(19) United States
(12) Patent Application Publication
(10) Pub. No.: US 2008/0082146 A1
(43) Pub. Date: Apr. 3, 2008

(54) TEMPERATURE COMPENSATION FOR ANALOG CIRCUITS IN IMPLANTABLE MEDICAL DEVICE

(75) Inventors: Rajesh Krishan Gandhi, Woodbury, MN (US); Paul J. McNamee, St. Paul, MN (US); Jacob M. Ludwig, Isanti, MN (US); Geoffrey Weinberg, Maple Grove, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc, St. Paul, MN (US)

(21) Appl. No.: 11/863,630

(22) Filed: Sep. 28, 2007

Related U.S. Application Data
(60) Provisional application No. 60/827,625, filed on Sep. 29, 2006.

(51) Int. Cl. A61N 1/00

(52) U.S. Cl. 607/60

(57) ABSTRACT

Temperate compensation is provided to analog circuits used in implantable medical devices. In various embodiments, temperature compensation is applied to improve calculation of battery characteristics, improve telemetry, and/or reduce battery self-discharge.

### CROSS-REFERENCE TO RELATED APPLICATIONS

This application claims the benefit of U.S. Provisional Application Ser. No. 60/827,625, filed Sep. 29, 2006 under 35 U.S.C. § 119(e) which is incorporated herein by reference in its entirety.

### FIELD OF THE INVENTION

The present application relates generally to analog circuits including at least one temperature sensor.

### BACKGROUND

The ever increasing capabilities of electronic circuitry have provided many beneficial uses. One important benefit involves the ability of physicians to improve the health of a patient using an implantable medical device (IMD) to monitor and regulate functions within the human body. IMD's rely heavily on complex, application specific circuitry. In many cases, analog circuitry is advantageous for use in an IMD. Some analog circuitry and functions, however, are sensitive to the temperature of the environment in which the IMD is manufactured, used, calibrated and stored. Often, an IMD is subject to varying environmental conditions. For example, during manufacture of an IMD, the environment temperature is typically room temperature. However, the storage environment for an IMD manufacture can vary from −20C to 70C. At low temperatures portions of an IMD suffer significant losses in accuracy. Furthermore, some portions of an IMD cease to function altogether. An example of inaccuracy is that battery voltage is currently calibrated at room temperature with additional bench data used to extrapolate the voltage at body temperature. Therefore, there is a need in the art to provide analog circuitry capable of compensating for changing temperature environments.

### SUMMARY

Method and apparatus for providing temperate compensation to analog circuits used in implantable medical devices is disclosed herein. Temperature compensation is applied to a battery voltage measurement to improve calculation of battery management characteristics. Also, temperature compensation is applied to a MHz oscillator trim to improve telemetry of an implantable medical device. Further, temperature compensation is applied to reduce battery self-discharge.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 is an illustration of an embodiment of temperature compensation for analog circuits used in implantable medical devices.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an” “one”, or “various” embodiments in this disclosure are not necessarily to the same embodiment, and such references contemplate more than one embodiment. The following detailed description provides examples, and the scope of the present invention is defined by the appended claims and their legal equivalents.

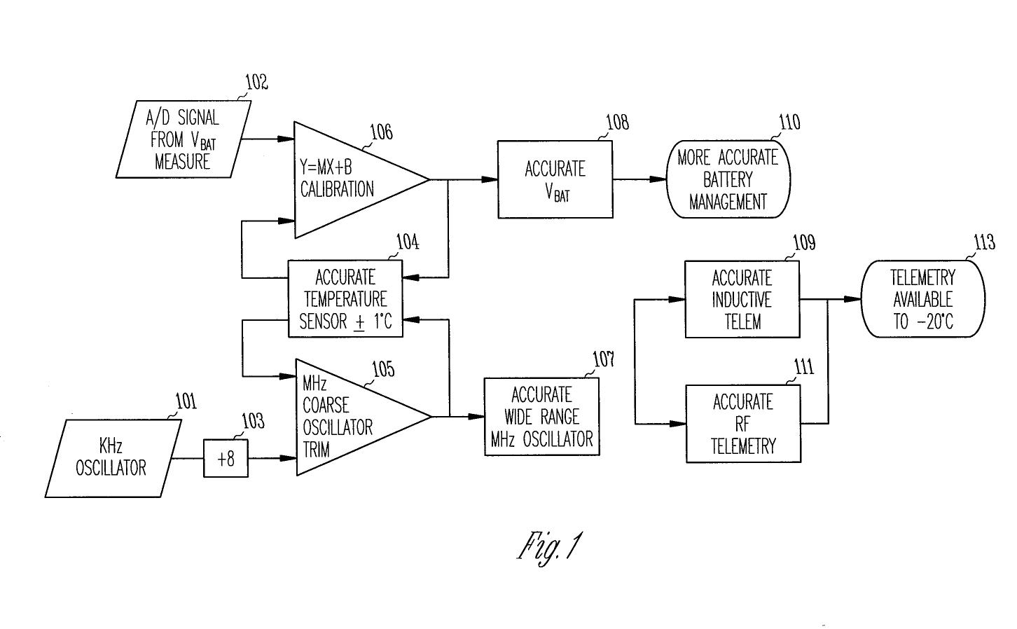

FIG. 1 illustrates various uses of temperature compensation for analog circuits used in implantable medical devices. In one use, temperature compensation is used to improve the accuracy of battery management. At 102, a signal representing a battery voltage measurement is determined. The signal representing a battery voltage measurement is determined using an analog-to-digital converter and a voltage reference, both of which are effected by temperature. Next, at 104, temperature sensor circuitry provides a measurement of temperature. At 106, the battery voltage measurement is calibrated using the measurement of temperature. At 108, an accurate measurement of battery voltage is provided. At 110, this accurate battery voltage measurement is used to provide more accurate battery management of an implantable medical device. Accurate battery voltage measurement is used to improve the accuracy of life phase triggering such as elective replacement interval, end of life, and other life phase measurements. Using this accurate battery voltage measurement improves the longevity of an implantable medical device battery by as much as 5%. This could mean a longevity improvement of weeks or months.

Still referring to FIG. 1, the use of temperature compensation for analog circuits is used to improve the telemetry, or communicative function, of an implantable medical device. The IMD may include a MHz oscillator. The accuracy of telemetry relies in part on the accuracy of a MHz oscillator. At 101, a KHz oscillator provides a signal. Next, at 103, this signal is divided. In this particular example, the signal is divided by 8. At 104, temperature sensor circuitry provides a measurement of temperature. At 105, the temperature signal and the divided KHz oscillator signal are provided to a MHz coarse oscillator trim. The MHz coarse oscillator trim adjusts the trim of the oscillator. In addition, the MHz coarse oscillator takes environmental temperature sensed by the temperature sensor into account to improve the accuracy of the trim adjustment. At 107, an accurate wide range MHz oscillator is provided by the MHz coarse oscillator trim.

At 109, inductive telemetry uses the accurate wide range MHz oscillator to improve telemetry. Inductive telemetry needs accuracy within 1%. At 111, radio frequency telemetry uses the accurate wide range MHz oscillator to improve telemetry. Radio frequency telemetry requires less accuracy, but the spectrum can drift as a function of temperature. Transmitting radio frequency signals outside an allocated spectrum is forbidden by the Federal Communications Commission and other regulatory bodies.

Fine trim actively monitors and corrects the MHz oscillator at higher temperatures. However, below 0C, fine trim range is exhausted and the MHz oscillator cannot be trimmed. The MHz oscillator could be triggered to update the coarse trim. If the MHz oscillator is triggered to update the coarse trim, at 113, both inductive and radio frequency telemetry are improved such that they are available for temperatures as low as −20C.

Temperature compensation for analog electronics is also used to reduce IMD battery self-discharge. Battery self-discharge occurs at elevated storage temperatures, and can effect the longevity of a battery. Battery self-discharge may be improved using a coulometer in communication with temperature measurement circuitry to compensate for pre-implant high self-discharge.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A method, comprising:
measuring the temperature of an implantable medical device environment;
measuring the battery voltage of a battery using an analog-to-digital converter and a voltage reference; and
using the measurement of temperature to compensate the battery voltage to provide an accurate battery voltage to improve the accuracy of life phase triggering.

2. A method, comprising:
measuring the temperature of an implantable medical device environment;
providing a MHz oscillator signal;
coarse trimming the MHz oscillator signal using the measured temperature to provide an accurate oscillator signal; and
using the accurate oscillator signal to improve the accuracy of inductive or RF telemetry of an implantable medical device.
